(19) United States
(12) Patent Application Publication
(10) Pub. No.: US 2023/0288378 A1
(43) Pub. Date: Sep. 14, 2023

(54) BUFFER ROD, DEVICE AND SYSTEM

(71) Applicants: Navin Sakthivel, Spring, TX (US); Aaron Avagliano, Tomball, TX (US); Baskaran Ganesan, Acton, MA (US); Wei Chen, Sugar Land, TX (US)

(72) Inventors: Navin Sakthivel, Spring, TX (US); Aaron Avagliano, Tomball, TX (US); Baskaran Ganesan, Acton, MA (US); Wei Chen, Sugar Land, TX (US)

(73) Assignee: Baker Hughes Oilfield Operations LLC, Houston, TX (US)

(21) Appl. No.: 17/494,478

(22) Filed: Oct. 5, 2021

(51) Int. Cl. G01N 29/28; G01N 29/02; G01N 33/00; G10K 11/02

(52) U.S. Cl. CPC G01N 29/28; G01N 29/02; G01N 33/005; G10K 11/02; G01N 29/2437

(57) ABSTRACT

A buffer rod for an acoustic measurement device including a ceramic nonmetallic body, a window plate disposed at one longitudinal end of the body, an opposite end of the body configured to interact with a transducer.

### BACKGROUND

Acoustic impedance matching is important in any acoustic measurement system to reduce scattering losses and improve signal conduction. This is particularly important in systems where a volume to be measured, whether that be gas, liquid or solid exhibits an acoustic impedance of significant difference than a piezoelectric crystal used as a transducer in the system. Such systems are ubiquitous in industry and while efforts have been made regarding the forgoing, alternatives and improvements are always well received.

### SUMMARY

An embodiment of a buffer rod for an acoustic measurement device including a ceramic nonmetallic body, a window plate disposed at one longitudinal end of the body, an opposite end of the body configured to interact with a transducer.

### BRIEF DESCRIPTION OF THE DRAWINGS

The following descriptions should not be considered limiting in any way. With reference to the accompanying drawings, like elements are numbered alike:

FIG. 1 is a schematic view of a buffer rod and associated components as disclosed herein; and

FIG. 2 is a schematic view of a processing system employing the buffer rod as disclosed herein.

### DETAILED DESCRIPTION

A detailed description of one or more embodiments of the disclosed apparatus and method are presented herein by way of exemplification and not limitation with reference to the Figures.

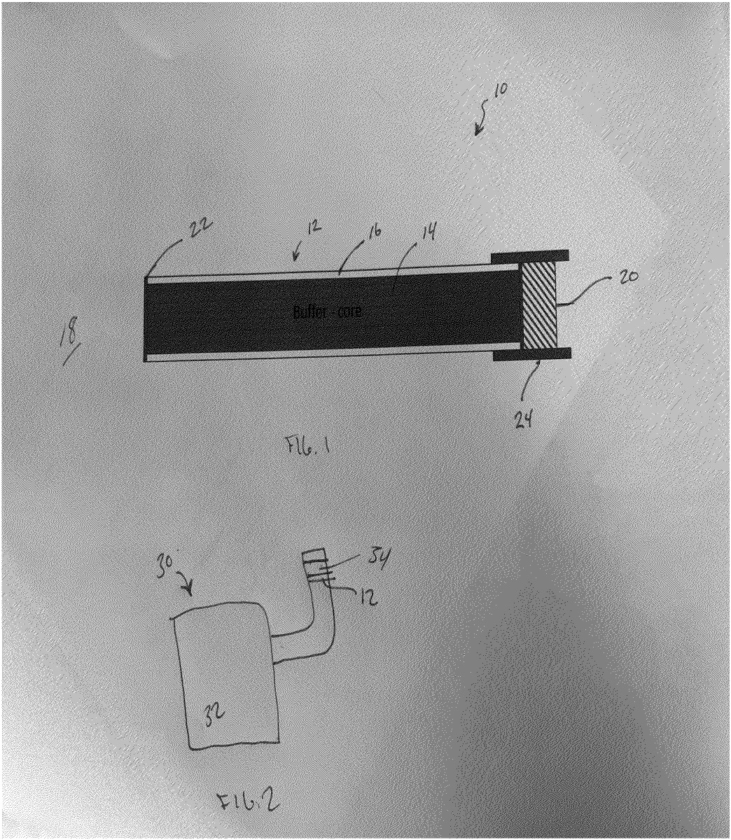

Referring to FIG. 1, a system 10 including an acoustic buffer rod 12 is illustrated. The rod 12 possesses a core 14 and optionally a buffer tube 16 about the core 14. The rod 12 is configured to extend from a zone to be measured 18 to a transducer 20 such as a piezoelectric crystal. The rod 12 is configured in embodiments to impedance match alone or to impedance match along with thermally protect the transducer 20. The rod 12 includes a plate window 22 attached to or formed as a part of the rod 12 to protect the rod 12 from a measurement environment that may be a very hot gas environment such as for example a flare gas. Attachment may be by welding in embodiments and may be by deposition in an additive manufacturing process in embodiments. The window plate is fluid impermeable and may be the same or a different material from the rod 12. At an opposite end of rod 12 is an interface 24 that is configured to provide operable contact with the transducer 20.

Material of the rod 12 comprises a nonmetallic ceramic material, one example of whish is Alumina. Various of the stated class of materials have acoustic and or thermally insulative properties (e.g. 200° F. to 800° F.), with alumina exhibiting both. In embodiments, 35-40 MRayls is desirable. By employing nonmetallic ceramic materials for the rod 12, the rod may be structurally shorter due to enhanced thermal insulative properties over the closest prior art, which employs stainless steel material.

Alumina and similar materials useful in connection with this disclosure include functionally graded materials. Functional grading of alumina may be effected by additive manufacturing processes such as binder-jetting, Direct Ink Writing, Direct Light Printing, or Material Jetting, for example. The functional grading of the material allows for tuning of the density of the material and thereby affecting in a desirable manner the acoustic impedance of the resulting rod 12. Materials used for the core 14 and tube 16 may be the same or different in various embodiments. Thickness of the tube 16 will be dictated by the frequency of the signal that will be propagated by the rod 12 to the transducer 20. Frequencies contemplated include 25-500 kiloHertz (KHz) for gas measurement and 250 KHz to 2 megaHertz (MHz) for liquid measurements.

Unengineered alumina possesses an acoustic impedance of 41-46 MRayls and an associated speed of sound of 10550 meters per second m/s however, when engineered as taught herein, the acoustic impedance is 20-41 MRayl and the speed of sound in the material is between 1000-12000 m/s. Lower ranges of acoustic impedance and speed of sound is technically possible and could be achieved but this may compromise the signal strength.

For density, the tube 16 in an embodiment comprises a density of 3.85-3.96 g/cc and the core 14 comprises a density of 2-3.95 g/cc. The core 14 may comprise a number of individual structures or a porous or lattice monolithic structure, as desired. The structures are employed to accomplish a functional gradient of material density or engineered material density, and hence achieve a function gradient or engineered acoustic impedance such that the structure is an acoustic waveguide.. Suitable porous alumina may be produced through admixture with sacrificial inclusions (such as hydrocarbons, plastics, low melt metals, etc.) that are later removed by heat or other removal process to leave porous alumina. Alternatively, porous alumina may be produced through a hybrid additive manufacturing process where alumina component and the sacrificial material component are both printed materials. In such hybrid process, the printed material (made of both components) would then undergo the same removal processes as the former example to create porosity in the Alumina.

Further in the case of Alumina, other properties created in the subject alumina to enhance performance in connection with the measurement of fluids such as flare gas include:

[symbol]

Referring to FIG. 2, a fluid processing system 30 is illustrated. System 30 includes a structure 32 that processes fluids such as flare gas, hydrogen etc. in liquid or gaseous form. A measurement subsystem 34 is fluidly connected to the structure 32 such that fluid therefrom maybe measured by subsystem 34. Subsystem 34 herein includes the system 10 described above, with the buffer rod 12 as described herein disposed between the target fluid and the rest of the system 10.

Set forth below are some embodiments of the foregoing disclosure:

Embodiment 1: A buffer rod for an acoustic measurement device including a ceramic nonmetallic body, a window plate disposed at one longitudinal end of the body, an opposite end of the body configured to interact with a transducer.

Embodiment 2: The rod as in any previous embodiment wherein the body is thermally insulative.

Embodiment 3: The rod as in any previous embodiment wherein the body is acoustically conductive.

Embodiment 4: The rod as in any previous embodiment wherein the body comprises a core.

Embodiment 5: The rod as in any previous embodiment wherein the core is of porous structure.

Embodiment 6: The rod as claimed in claim 1 wherein the core is of lattice structure.

Embodiment 7: The rod as in any previous embodiment wherein the core is surrounded by a tube.

Embodiment 8: The rod as in any previous embodiment wherein the tube comprises a different material than the core.

Embodiment 9: The rod as in any previous embodiment wherein the tube comprises the same material as the core.

Embodiment 10: The rod as in any previous embodiment wherein the body comprises alumina.

Embodiment 11: The rod as in any previous embodiment wherein the alumina is processed using at least one of the additive manufacturing processes of Binder Jetting, Material Jetting, or Direct Ink Writing.

Embodiment 12: The rod as in any previous embodiment wherein the window plate is of the same material as the body.

Embodiment 13: The rod as in any previous embodiment wherein the window plate is of a different material than the body.

Embodiment 14: An acoustic measurement device including a transducer, a buffer rod as in any previous embodiment in operable contact with the transducer.

Embodiment 15: The device as in any previous embodiment wherein the transducer is a piezo electric crystal.

Embodiment 16: A method for producing a buffer rod as in any previous embodiment comprising applying a feed material to a build plate.

Embodiment 17: The method as in any previous embodiment further comprising manipulating a feed material with a recoater, roller, nozzle, extruder, or deposition head in an additive manufacturing process.

Embodiment 18: A processing system including a fluid measurement subsystem, the subsystem comprising the buffer rod as in any previous embodiment.

Embodiment 19: The system as in any previous embodiment wherein the fluid measurement subsystem is a flare gas measurement subsystem.

Embodiment 20: The system as in any previous embodiment wherein the fluid measurement subsystem is a hydrogen measurement subsystem.

The use of the terms “a” and “an” and “the” and similar referents in the context of describing the invention (especially in the context of the following claims) are to be construed to cover both the singular and the plural, unless otherwise indicated herein or clearly contradicted by context. Further, it should be noted that the terms “first,” “second,” and the like herein do not denote any order, quantity, or importance, but rather are used to distinguish one element from another. The terms “about”, “substantially” and “generally” are intended to include the degree of error associated with measurement of the particular quantity based upon the equipment available at the time of filing the application. For example, “about” and/or “substantially” and/or “generally” can include a range of ± 8% or 5%, or 2% of a given value.

The teachings of the present disclosure may be used in a variety of well operations. These operations may involve using one or more treatment agents to treat a formation, the fluids resident in a formation, a wellbore, and / or equipment in the wellbore, such as production tubing. The treatment agents may be in the form of liquids, gases, solids, semi-solids, and mixtures thereof. Illustrative treatment agents include, but are not limited to, fracturing fluids, acids, steam, water, brine, anticorrosion agents, cement, permeability modifiers, drilling muds, emulsifiers, demulsifiers, tracers, flow improvers etc. Illustrative well operations include, but are not limited to, hydraulic fracturing, stimulation, tracer injection, cleaning, acidizing, steam injection, water flooding, cementing, etc.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. A buffer rod for an acoustic measurement device comprising:
a ceramic nonmetallic body;
a window plate disposed at one longitudinal end of the body, an opposite end of the body configured to interact with a transducer.

2. The rod as claimed in claim 1 wherein the body is thermally insulative.

3. The rod as claimed in claim 1 wherein the body is acoustically conductive.

4. The rod as claimed in claim 1 wherein the body comprises a core.

5. The rod as claimed in claim 1 wherein the core is of porous structure.

6. The rod as claimed in claim 1 wherein the core is of lattice structure.

7. The rod as claimed in claim 4 wherein the core is surrounded by a tube.

8. The rod as claimed in claim 7 wherein the tube comprises a different material than the core.

9. The rod as claimed in claim 7 wherein the tube comprises the same material as the core.

10. The rod as claimed in claim 1 wherein the body comprises alumina.

11. The rod as claimed in claim 10 wherein the alumina is processed using at least one of the additive manufacturing processes of Binder Jetting, Material Jetting, or Direct Ink Writing.

12. The rod as claimed in claim 1 wherein the window plate is of the same material as the body.

13. The rod as claimed in claim 1 wherein the window plate is of a different material than the body.

14. An acoustic measurement device comprising:
a transducer;
a buffer rod as claimed in claim 1 in operable contact with the transducer.

15. The device as claimed in claim 14 wherein the transducer is a piezo electric crystal.

16. A method for producing a buffer rod as claimed in claim 1 comprising applying a feed material to a build plate.

17. The method as claimed in claim 16 further comprising manipulating a feed material with a recoater, roller, nozzle, extruder, or deposition head in an additive manufacturing process.

18. A processing system including a fluid measurement subsystem, the subsystem comprising the buffer rod as claimed in claim 1.

19. The system as claimed in claim 18 wherein the fluid measurement subsystem is a flare gas measurement subsystem.

20. The system as claimed in claim 18 wherein the fluid measurement subsystem is a hydrogen measurement subsystem.
